(19) United States
(12) Patent Application Publication
(10) Pub. No.: US 2010/0145220 A1
(43) Pub. Date: Jun. 10, 2010

(54) FEEDBACK DEVICE

(75) Inventor: Paulette Margarete van Vliet, Leicestershire (GB)

Correspondence Address: ANDRUS, SCEALES, STARKE & SAWALL, LLP, 100 EAST WISCONSIN AVENUE, SUITE 1100, MILWAUKEE, WI, 53202, US

(73) Assignee: THE UNIVERSITY OF NOTTINGHAM

(21) Appl. No.: 12/532,685

(22) PCT Filed: Mar. 20, 2008
(86) PCT No.: PCT/GB08/01087

§ 371 Date: Feb. 10, 2010

(30) Foreign Application Priority Data
Mar. 23, 2007 (GB) 0705564.3

(51) Int. Cl. A61B 5/053; A61B 5/11

(52) U.S. Cl. 600/547; 73/865.4; 600/595

(57) ABSTRACT

A feedback device is described comprising sensing means operable to detect a first body part movement and a second body part movement. A timer, associated with said sensing means, is provided for measuring the time lapsed between detection of the first body part movement and detection of the second body part movement. A communication means is also provided for relaying information concerning the lapsed time to a user.

### FIELD OF THE INVENTION

This invention relates to a feedback device. Particularly, but not exclusively, the invention relates to a feedback device that can be used in the rehabilitation of the reach-to-grasp function in stroke patients.

### BACKGROUND TO THE INVENTION

Each year, over 130,000 people in the UK suffer from a stroke. A stroke is a condition that results in damage to the brain as a result of either a loss of blood to the brain or acute bleeding in the brain. Various neurological deficits can arise as a result of a stroke. In particular, hemiparesis, which denotes muscle weakness or partial paralysis down one side of the body, often occurs. This impairs use of the upper and lower limbs and affects balance. Deficits affecting the upper limb are a particular problem, with only approximately 56% of stroke survivors regaining useful function in a hemiparetic arm. Consequently, the degree of arm motor impairment is often seen as the most influential factor in determining the well being of a patient one year after a stroke.

Research has shown that hand and arm coordination is a particular problem associated with upper limb rehabilitation in patients who have suffered from a stroke in the area of the brain supplied by the middle cerebral artery. Poor hand-arm coordination can result in an inaccurate grasp. This can make simple everyday activities, such as picking up a glass of water, extremely difficult.

In respect of a so-called reach-to-grasp movement, it has been observed that patients who have suffered from middle cerebral artery stroke tend to exhibit a much lower correlation between the beginnings of the grasp function (the hand opening and then closing on an object) and the transport function (the change in position of the wrist as it moves towards the object), than for healthy people. The correlation of this pair of events is thought to be key to the effective temporal coordination of the whole reach-to-grasp movement, since if the components begin together there is more chance of maintaining coordination throughout the remainder of the movement.

Routine rehabilitation of the reach-to-grasp movement in stroke patients frequently involves a physiotherapist observing a patient's movement and monitoring their progress as they repeatedly practice the movement, and giving the patient feedback to improve the next attempt. The effectiveness of this technique therefore relies on the ability of the physiotherapist to judge the correlation of the grasp and reach functions by eye and to notice when an improvement is made. This can be extremely difficult to observe with only significant improvements being noticeable. In addition, it is difficult for a patient to practice the correlation of the movement in their own time since it is extremely difficult for them to judge the results themselves.

It is therefore an aim of the present invention to address the above issues.

### SUMMARY OF THE INVENTION

According to a first aspect of the present invention there is provided a device comprising sensing means operable to detect a first body part movement and a second body part movement; a timer associated with said sensing means and capable of measuring the time lapsed between detection of the first body part movement and detection of the second body part movement; and a communication means for relaying information concerning the lapsed time to a user.

Thus, with the sensing means arranged on appropriate body parts, the device of the first aspect of the present invention can be used to accurately measure the time lapsed between the beginning of a grasp function and the beginning of a reach or transport function during a reach-to-grasp movement. Communication of the lapsed time to the user enables instant feedback on the correlation of the two events and therefore on their coordination at the start of the movement. It also allows successive movements to be compared. The device may be particularly useful to physiotherapists attempting to retrain the reach-to-grasp movement in stroke patients since it enables the grasp and transport functions to be practised together so that coordination between these components can be rehearsed and improved. This may be much more beneficial than practising separate exercises for example for the shoulder, elbow, wrist and hand movements. In addition, it has been observed that in healthy subjects the events are more tightly coupled when undertaking faster movements. However, this phenomenon does not occur in stroke patients. Accordingly, the device of the present invention can be used to provide feedback on reach-to-grasp exercises that are practiced at different speeds in order to enable practice of tighter coupling in faster movements.

The first body part movement and the second body part movement may relate, respectively, to first and second movements of the same body part or to one movement of one body part and another movement of another body part.

The sensing means may be configured to detect one or more of the following: the location of the first and/or second body parts, the speed of the first and/or second body part movements, the direction of the first and/or second body part movements, and the acceleration/deceleration of the first and/or second body part movements. The sensing means may also be configured to detect pre-programmed events such as changes in the speed/direction/acceleration/deceleration of the first and/or second body part movements. Accordingly, it will be understood that in embodiments of the present invention, the timer may record the time lapsed between an event triggered during the acceleration of a first body part movement and an event triggered during the acceleration of a second body part movement.

The information relayed by the communication means may be the actual time lapsed and/or a more general indication of the time lapsed, for example, by displaying whether the lapsed time was ‘slow’, ‘average’ or ‘fast’.

The device may also be configured to communicate the temporal order of the movements detected.

The device may include an indication means to indicate its operational status to the user. The indication means may be configured to indicate one or more of the following states: an initialisation state, a ready state, a timing state, and a timing complete state.

The sensing means may comprise one or more touch, pressure, acceleration/deceleration or location sensors.

The sensing means may include a first sensor operable to detect the first body part movement and a second sensor operable to detect the second body part movement.

In a particular embodiment, the first sensor may be configured to detect the opening of a user's hand (such as would occur at the beginning of a grasp movement) and the second sensor may be configured to detect the displacement of a user's wrist from a starting position.

In an embodiment of the invention, the first and second sensors may be touch sensors. The touch sensors may be contact electrodes configured to monitor the electrical conductivity of a user's body through the surface of the user's skin. The first sensor may comprise a thumb electrode that is metallised on its outer surface such that placing a forefinger into contact with the thumb electrode initiates a grasp-closed condition. The second sensor may comprise a wrist electrode in the form of a metallic base plate configured to detect gross contact of a user's lower arm or wrist when at rest. Preferably, in this embodiment, the device further includes a body grounding means to hold the electrical potential of a user's body at a local system ground. The body grounding means may be in the form of a wristband to be worn by a user whenever the device is in use.

In the above embodiment, an onset of motion is detected by instant of loss of contact of either the thumb electrode or the wrist electrode. This triggers the timer to start timing. Subsequent loss of contact of the remaining thumb or wrist electrode triggers the timer to stop timing and the resulting lapsed time is communicated to the user. The order of the events (i.e. grasp first or second) may also be communication to the user.

The communication means may comprise a visual display and/or a loudspeaker.

The device may have a timing resolution of 1 ms.

The device may be configured for battery and/or mains operation.

According to a second aspect of the present invention there is provided a method of determining the time lapsed between related body part movements during a coordinated activity, the method comprising:

arranging a sensing means to be able to detect a first body part movement and a second body part movement;

starting a timer upon detection of the first body part movement and stopping the timer upon detection of the second body part movement; and

relaying information concerning the lapsed time to a user.

### BRIEF DESCRIPTION OF THE DRAWINGS

FIG. 1 illustrates schematically an embodiment of a device according to the present invention;

FIGS. 2 to 9 show various circuit diagrams illustrating the logic employed in the device of FIG. 1; more specifically,

FIG. 2 shows the initialisation and status control circuit;

FIG. 3 shows how the device determines whether a wrist contact or a grasp contact is on or off;

FIG. 4 shows how the device determines whether to proceed to a ready status or a timing status and also shows how the device memorises the temporal order of events;

FIG. 5 shows how the device determines whether to proceed to a timing complete and counter reset status;

FIG. 6 shows a voltage stabilised power supply specifically for the timing circuit;

FIG. 7 shows the timing circuit for the device;

FIG. 8 shows how the device displays the appropriate status; and

FIG. 9 shows the charging circuit for the device and the system battery power source.

### DETAILED DESCRIPTION OF CERTAIN EMBODIMENTS

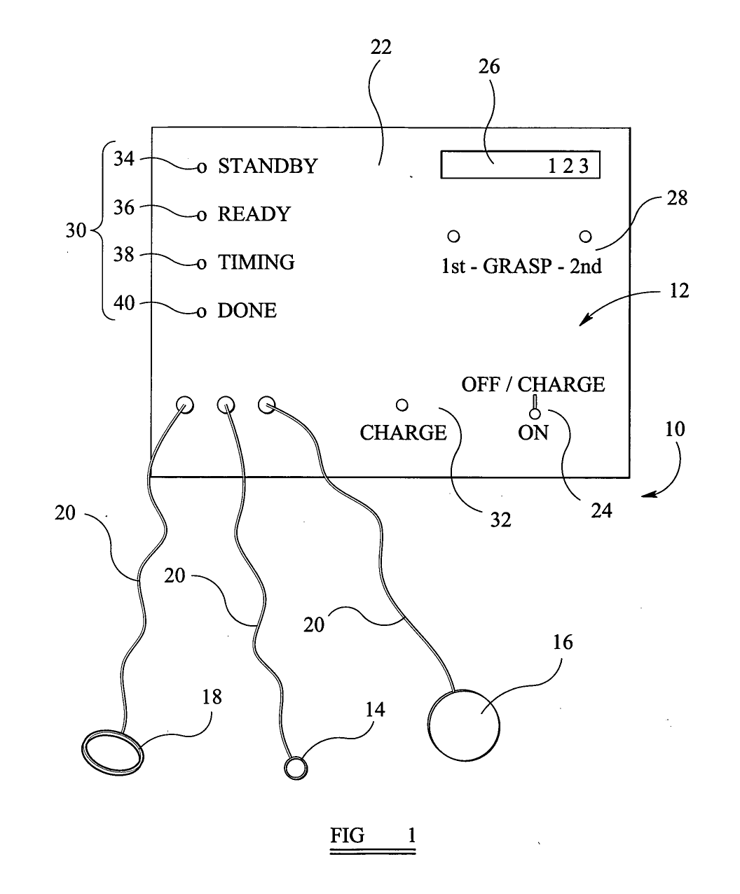

With reference to FIG. 1, there is illustrated a device 10 according to the present invention. The device 10 comprises an electronic control unit 12, a first sensor in the form of a thumb electrode 14, a second sensor in the form of a wrist electrode 16 and a body grounding means in the form of a wristband 18. The thumb electrode 14, wrist electrode 16 and wristband 18 are, respectively, connected by wires 20 to the control unit 12. The thumb electrode 14 comprises a cylindrical tube or ring with an insulated inner surface and a metallised outer surface. The ring is of a size to comfortably fit over an adult thumb. The wrist electrode 16 comprises a circular metal base plate of a size to accommodate an adult user's wrist and hand when the hand is in a loose fist. The wristband 18 is an elasticated bracelet designed to be placed over an adult user's hand and to rest snugly around their wrist. The wristband 18 has a metallic contact on its inner surface.

The control unit 12 includes a front panel 22 with an on/off switch 24, a timer display panel 26, a movement order display 28, status indication means 30, a charge socket 32, and associated indicia. The timer display panel 26 is a LCD screen configured to display times in milliseconds. The movement order display 28 comprises two LEDs respectively configured to light up to indicate whether the grasp function of a reach-to-grasp movement was first or second in relation to the transport function—as measured through the thumb electrode 14 and wrist electrode 16 and described in more detail later. The status indication means 30 comprises a ‘Standby’ LED 34 configured to light up when the system is powering up, a ‘Ready’ LED 36 configured to light up when the device is ready for the user to begin a reach-to-grasp movement, a ‘Timing’ LED 38 configured to light up after the first contact is broken and the device is timing, and a ‘Done’ LED 40 configured to light up after the second contact is broken and the timing is complete.

To operate the device, a user should connect the wires 20 to the appropriate inputs on the control unit 12, position the wrist electrode 16 on a convenient surface over which the exercise will take place, place the wristband 18 on the patient's wrist (preferably, the wrist of the arm which is not being exercised), place the thumb electrode 14 on the patient's thumb (of the arm which is to be exercised), and switch the device 10 to ON. The device 10 is designed to operate entirely automatically from this point on. The device 10 will default to the ‘Standby’ condition as the unit is powering up and the ‘Standby’ LED 34 will illuminate. The status will remain until the user adopts the correct starting posture. The correct starting posture is with the hand, wrist or arm placed comfortably on the wrist electrode 16 and a finger (usually the forefinger) of the same hand touching the thumb electrode 14. When this position has been held steady for a set time (i.e. 3 seconds) the unit will be armed and will advance to the ‘Ready’ condition, the ‘Ready’ LED 36 being illuminated. At any time after the ‘Ready’ condition has been met the user can initiate the reach-to-grasp movement. Removal of the finger off the thumb electrode 14 or removal of the hand/wrist/arm off the wrist electrode 16 is detected by the control unit 12 as a loss of conductivity. The occurrence of either of the above initiates a timer in the control unit 12 to begin timing and the ‘Timing’ condition will be indicated by illumination of the ‘Timing’ LED 38. The occurrence of the subsequent further loss of contact stops the timer and the ‘Done’ condition will be indicated by illumination of the ‘Done’ LED 40. Thus, the interval between the first and second movements is measured and then displayed on timer display panel 26. The order of the movements is also detected and displayed to the user through the movement order display 28 (i.e. by illumination of the GRASP 1^{st} or 2^{nd} LED). The time and order data is conveniently displayed until the next ‘Ready’ condition is detected. The time being cleared to zero at this point and the order LEDs being switched off.

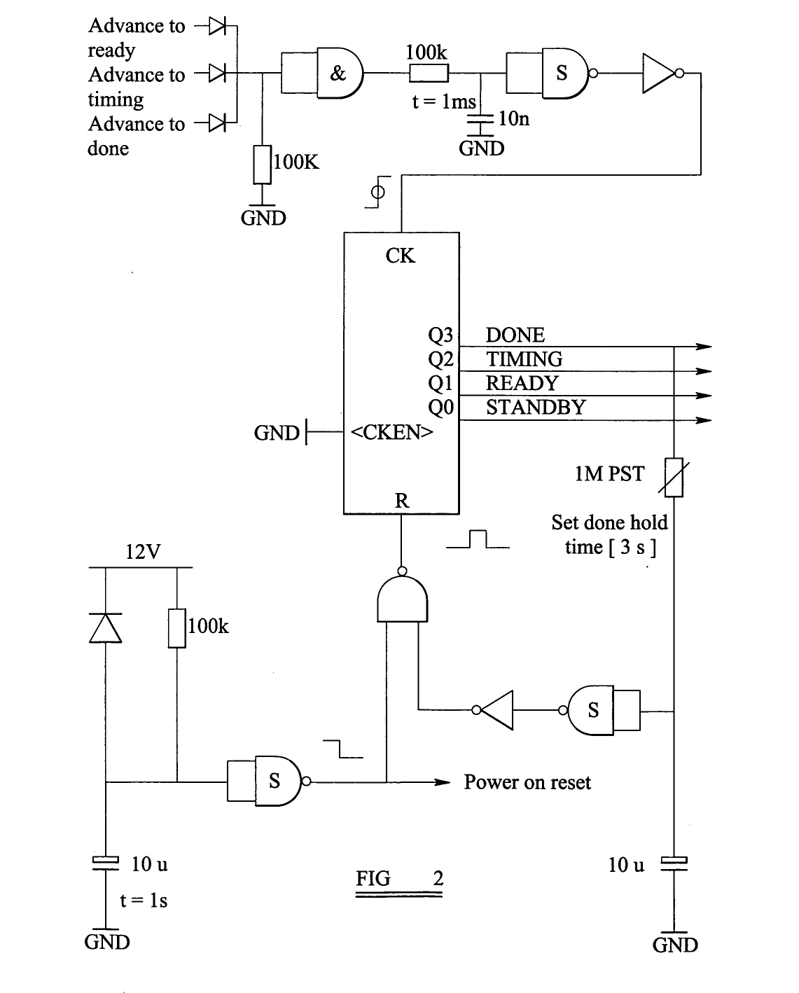

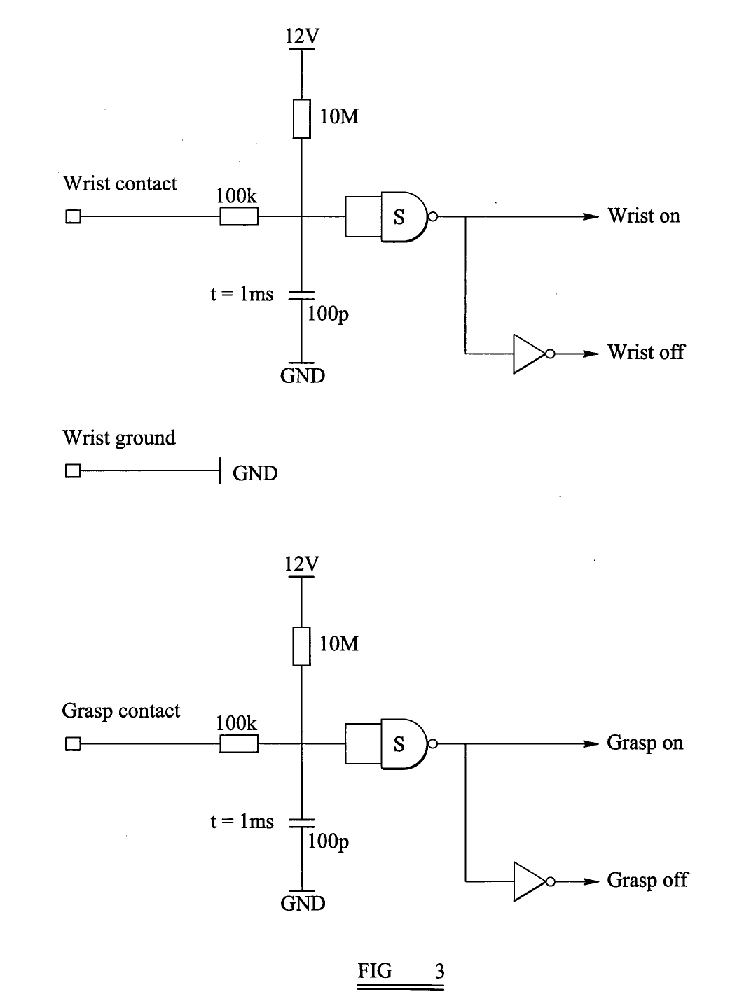

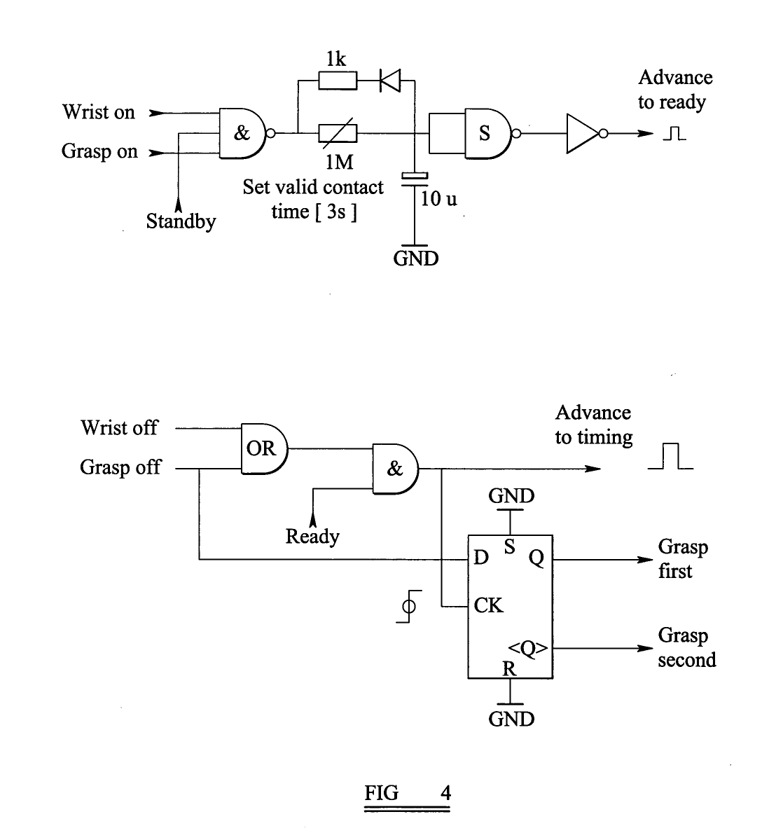

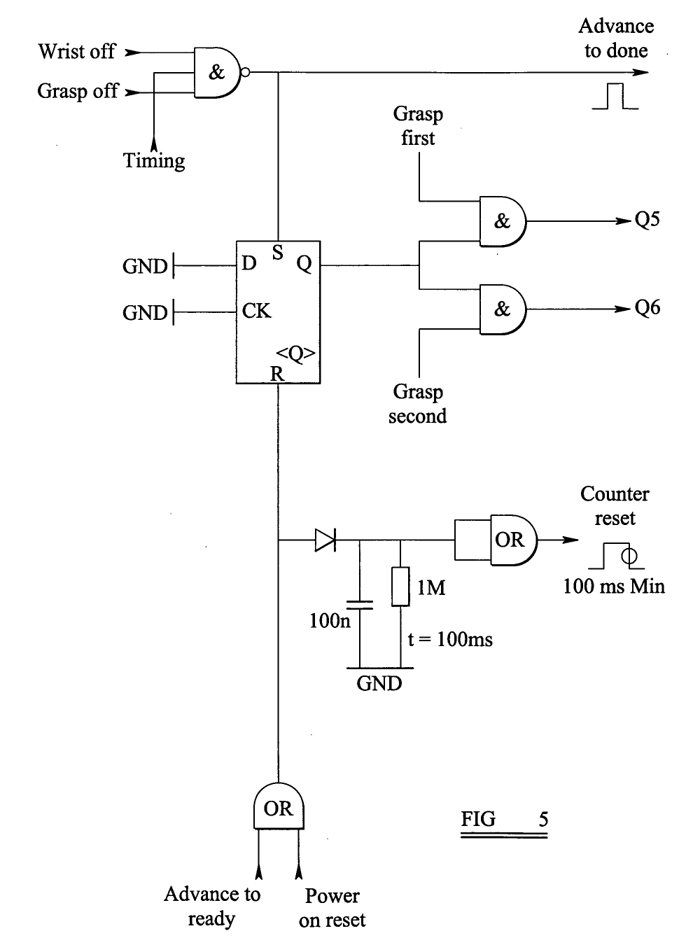

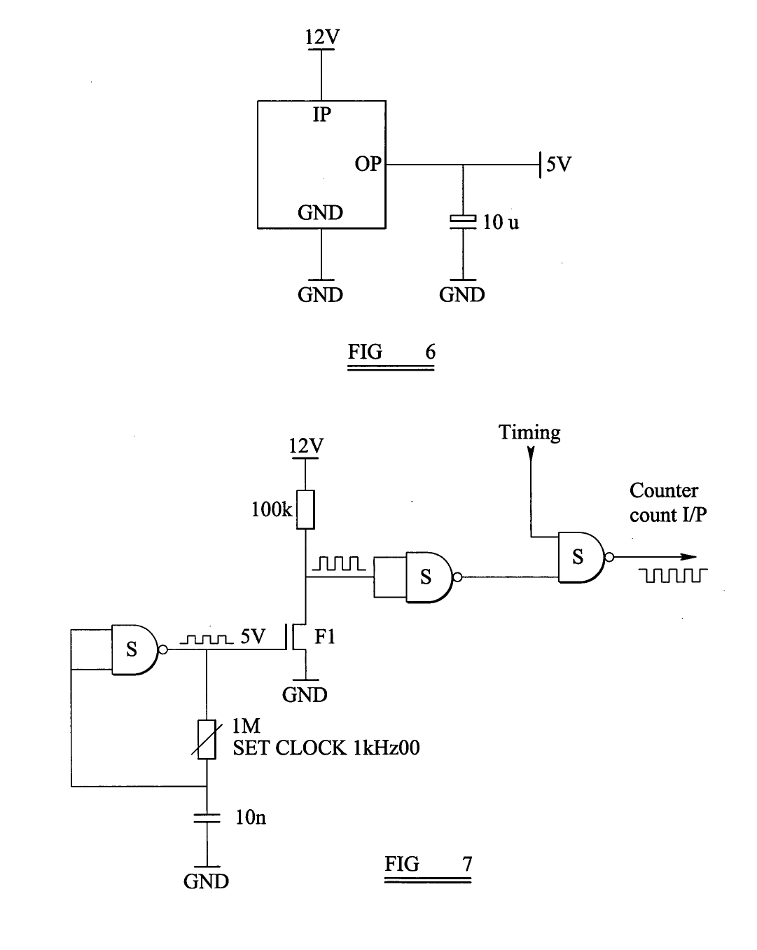

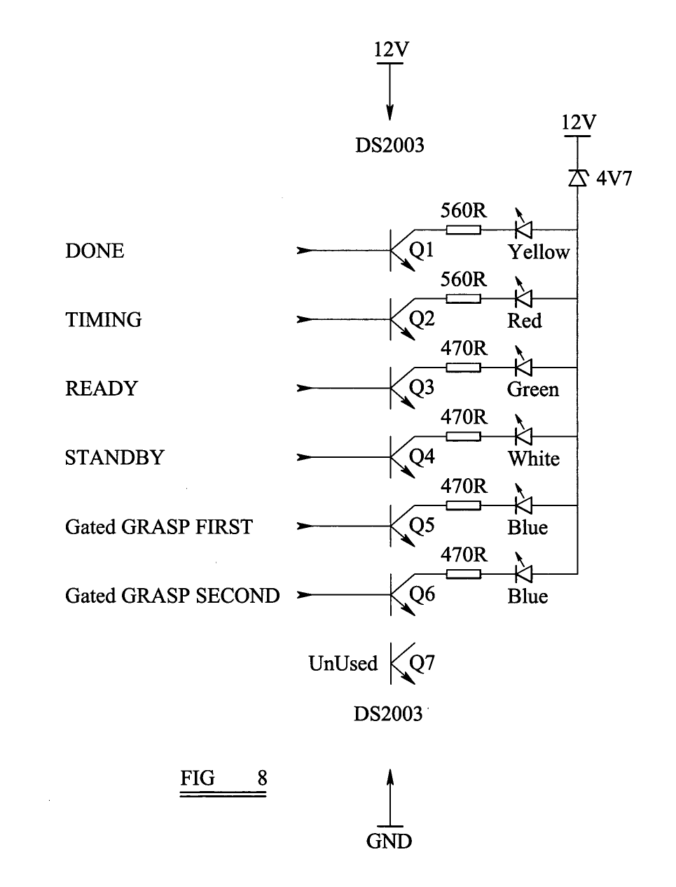

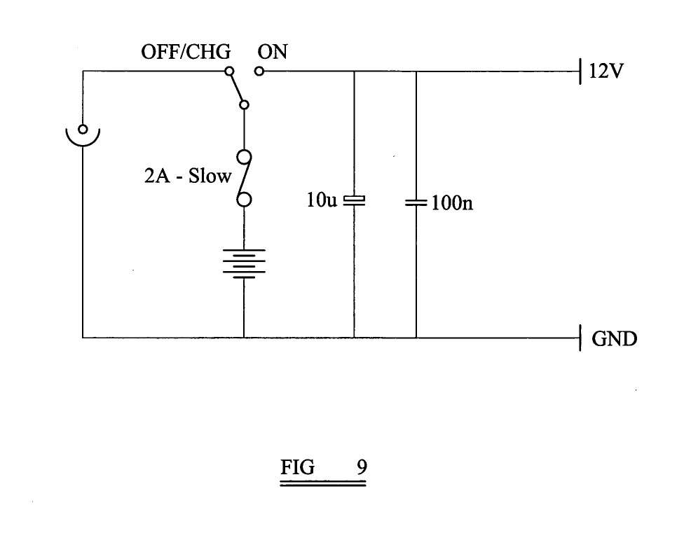

Circuit diagrams illustrating the logic employed in the above device 10 are shown in FIGS. 2 to 9. It is believed that the detailed functionality of these circuits will be apparent to those skilled in the art. In particular, FIG. 2 shows the initialisation and status control circuit employed in the control unit 12. FIG. 3 shows a circuit that enables the device to determine whether a wrist contact or a grasp contact is on or off. FIG. 4 shows a circuit that enables the device to determine whether to proceed to a ready status or a timing status and also shows how the device memorises the temporal order of events. FIG. 5 shows a circuit that enables the device to determines whether to proceed to a timing complete and counter reset status. FIG. 6 shows a voltage stabilised power supply specifically for the timing circuit. This maintains high timing accuracy under normally variable battery supply voltage. FIG. 7 shows the timing circuit for the device. FIG. 8 shows a circuit that enables the device to display the appropriate status and FIG. 9 shows the charging circuit for the device and the system battery power source.

It will be understood by those skilled in the art, that the functionality of the device 10 described above can be achieved in a number of ways. For example, although this particular device was based on discrete CMOS logic, a digital signal processor or microcontroller might be employed and various modifications may be made to the internal operation of the device without altering the external function.

The purpose of this particular device is to measure and display the biometric time interval between the onset of a hand grasp action and an associated arm motion, in either order. The timing is initiated on the first sign of contact loss from one of the touch sensors. The second sign of contact loss from the remaining touch sensor terminating the timing.

The device is fully automated and therefore easy to use. This is an important consideration since the device is intended to be used by patients who may have serious mental and/or physical deficiencies. The device also communicates the results of the exercise to the user in a simple yet informative way. The measured time can be noted and a user can attempt to ‘beat’ the previous attempt and therefore improve their coordination. The accurate feedback to the user is important in encouraging the user to practice the exercise and also allows even slight changes to be more easily noticed. A major advantage with the present device is that it can be used by patients on their own without the supervision of another person, such as a physiotherapist.

Intermittent or unreliable operation of the above device 10 may be encountered when a user has particularly dry skin. This can be remedied by moistening the various parts of the skin that are in contact with the sensors.

Intermittent or unreliable operation of the above device 10 may also be encountered when the contact between the skin and the thumb touch sensor is not sufficiently close. This can be remedied by using a smaller thumb electrode.

The device 10 described above contains rechargeable NiMH batteries. A fully charged battery will run the device for a full 30 hours. Low battery power will be indicated by a failure of the timer display panel 26 to glow bright green. A charger (not shown) has been developed to recharge the batteries of this device. To charge the device, the charger should be connected to the charge socket 32 and the on/off switch 24 switched to OFF (also labelled CHARGE). Conveniently, the charger is configured to indicate charging by illumination of a red LED and full charge by illumination of a green LED. A full recharge of the device will take approximately 5 hours. However, shorter charge times will permit partial or top-up charge.

An advantage of the particular embodiment of the invention described above is that as it employs superficial skin contact as the sensory technique, it creates an unobtrusive and ergonomic environment for the user. In addition, the automatic operational flow of the machine orchestrates the user into the desired starting position and leads the user through the exercise.

It is envisaged that a patient will wear the device whilst practising reach-to-grasp movements, initially under the guidance of a therapist but thereafter they may practice on their own. Ideally, the patient should practice for 30 minutes at a time, using the information to improve the synchronization of the two events.

The device may be used in a patient's home, or an inpatient or outpatient hospital or rehab setting.

It has been found that for a healthy person, the time between breaking two connections is from 0 to 40 ms. However, in stroke patients this time can be anything up to approximately 300 ms. It has also been found that the grasp connection is usually broken first by a healthy person but is often broken second by a stroke patient.

Although the above embodiment has employed touch sensors to detect the two movements other types of sensors may be used such as pressure sensors or location sensors.

A variant of the above embodiment may include additional features, for example to measure the reaction of the user to an instruction to reach-to-grasp. In this case, a buzzer may sound to serve as the instruction and the time lapsed between the buzzer sounding and the loss of the first contact can be recorded and displayed as the reaction time.

A further variant could be configured to also measure the maximum extent of hand opening during the grasp action.

A still further variant may employ an accelerometer, perhaps mounted on a user's wrist, to detect the acceleration experienced by the user's wrist during a reach part of an exercise.

It will be appreciated by persons skilled in the art that various modifications may be made to the above-described embodiments without departing from the scope of the present invention. For example, whilst the above discussion has been primarily concerned with the rehabilitation of stroke patients, the invention is equally applicable to the rehabilitation of other types of patients such as those with head injuries or other conditions that affect coordination. In addition, the device may be used in the training of a prosthetic or robotic body part. Furthermore, a device according to the present invention may be configured to provide feedback on other types of coordinated movement, for example, involving the lower limb.

1. A device comprising sensing means operable to detect a first body part movement and a second body part movement; a timer associated with said sensing means and capable of measuring the time lapsed between detection of the first body part movement and detection of the second body part movement; and a communication means for relaying information concerning the lapsed time to a user.

2. The device according to claim 1 wherein the first body part movement and the second body part movement relate, respectively, to first and second movements of the same body part.

3. The device according to claim 1 wherein the sensing means is configured to detect one or more of the following: the location of the first and/or second body parts, the speed of the first and/or second body part movements, the direction of the first and/or second body part movements, and the acceleration/deceleration of the first and/or second body part movements.

4. The device according to claim 1 wherein the sensing means is configured to detect changes in the speed, direction, acceleration, or deceleration of the first and/or second body part movements.

5. The device according to claim 1 wherein the timer is configured to record the time lapsed between an event triggered during the acceleration of a first body part movement and an event triggered during the acceleration of a second body part movement.

6. The device according to claim 1 wherein the information relayed by the communication means is the actual time lapsed.

7. The device according to claim 1 wherein the information relayed by the communication means comprises a general indication of the time lapsed.

8. The device according to claim 1 further configured to communicate the temporal order of the movements detected.

9. The device according to claim 1 further comprising an indication means to indicate its operational status to the user.

10. The device according to claim 9 wherein the indication means is configured to indicate one or more of the following states: an initialization state, a ready state, a timing state, and a timing complete state.

11. The device according to claim 1 wherein the sensing means comprises one or more touch, pressure, acceleration/deceleration or location sensors.

12. The device according to claim 1 wherein the sensing means comprises a first sensor operable to detect the first body part movement and a second sensor operable to detect the second body part movement.

13. The device according to claim 12 wherein the first sensor is configured to detect the opening of a user's hand and the second sensor is configured to detect the displacement of a user's wrist from a starting position.

14. The device according to claim 12 wherein the first and second sensors are touch sensors constituted by contact electrodes configured to monitor the electrical conductivity of a user's body through the surface of the user's skin.

15. The device according to claim 14 wherein the first sensor comprises a thumb electrode that is metallised on its outer surface such that placing a forefinger into contact with the thumb electrode initiates a grasp-closed condition.

16. The device according to claim 15 wherein the second sensor comprises a wrist electrode in the form of a metallic base plate configured to detect gross contact of a user's lower arm or wrist when at rest.

17. The device according to claim 14 wherein the device further includes a body grounding means to hold the electrical potential of a user's body at a local system ground.

18. The device according to claim 17 wherein the body grounding means is in the form of a wristband to be worn by a user whenever the device is in use.

19. The device according to claim 16 wherein an onset of motion is detected by instant of loss of contact of either the thumb electrode or the wrist electrode and this triggers the timer to start timing.

20. The device according to claim 19 wherein subsequent loss of contact of the remaining thumb or wrist electrode triggers the timer to stop timing and the resulting lapsed time is communicated to the user.

21. The device according to claim 1 wherein the communication means comprises a visual display and/or a loudspeaker.

22. The device according to claim 1 wherein the timer has a resolution of at least 1 ms.

23. A method of determining the time lapsed between related body part movements during a coordinated activity, the method comprising:
arranging a sensing means to be able to detect a first body part movement and a second body part movement;
starting a timer upon detecting of the first body part movement and stopping the timer upon detection of the second body part movement; and
relaying information concerning the lapsed time to a user.
